(19) United States
(12) Patent Application Publication
(10) Pub. No.: US 2019/0019397 A1
(43) Pub. Date: Jan. 17, 2019

(54) LAYERED DETECTOR DESIGN CONNECTED WITH SMARTPHONE BY EARPHONE TERMINAL

(71) Applicant: Honeywell International Inc., Morris Plains, NJ (US)

(72) Inventors: Bora Lee, Seoul (KR); YoungSoo Han, Seoul (KR)

(21) Appl. No.: 16/034,187

(22) Filed: Jul. 12, 2018

Related U.S. Application Data
(60) Provisional application No. 62/531,510, filed on Jul. 12, 2017.

(51) Int. Cl. G08B 21/16; G01N 33/00; H05K 5/02

(52) U.S. Cl. CPC G08B 21/16; G01N 33/0063; H01R 24/58; H05K 5/0247; H05K 5/0217

(57) ABSTRACT

Embodiments relate generally to systems and methods for detecting flammable gas. A method may comprise powering one or more sensor elements of a sensor module; contacting the one or more sensor elements with ambient air; detecting, by the sensor elements, one or more flammable gas within the ambient air; sending, via a 3.5 mm plug connector of the sensor module, the detected information from the sensor elements to a mobile device; receiving, by an application on the mobile device via a 3.5 mm plug input of the mobile device, the detected information from the sensor module; and displaying, via a user interface of the mobile device, at least a portion of the received information from the sensor module.

### CROSS-REFERENCE TO RELATED APPLICATIONS

The present application claims priority to U.S. Provisional Patent Application Ser. No. 62/531,510 filed Jul. 12, 2017 by Bora Lee, et al. and entitled “Layered Detector Design Connected with Smartphone by Earphone Terminal” which is incorporated herein by reference as if reproduced in its entirety.

### STATEMENT REGARDING FEDERALLY SPONSORED RESEARCH OR DEVELOPMENT

Not applicable.

### REFERENCE TO A MICROFICHE APPENDIX

Not applicable.

### BACKGROUND

Gas detectors may be carried by individuals throughout a hazardous environment and may detect gases in the environment. Gas detectors may be configured to alert a user and/or supervisor when a harmful gas or level of gas is detected. Gas detectors may also be configured to communicate sensed information to a mobile device.

### SUMMARY

In an embodiment, a sensor module may comprise a housing configured to allow airflow into the interior of the housing; one or more sensor elements configured to detect flammable gas; and a connector configured to connect to an input of a mobile device, and configured to send information to the mobile device.

In an embodiment, a communication system may comprise a sensor module comprising: a housing comprising a sintered filter; one or more sensor elements configured to detect flammable gas; and a 3.5 mm plug connector; a mobile device comprising; a 3.5 mm plug input configured to receive the 3.5 mm plug connector of the sensor module; a user interface; a processor; a memory; and an application stored by the memory that when executed by the processor is configured to receive, via the 3.5 mm plug input, detected information from the one or more sensor elements of the sensor module; and display, via the user interface, at least a portion of the received information from the sensor module.

In an embodiment, a method for detecting flammable gas may comprise powering one or more sensor elements of a sensor module; contacting the one or more sensor elements with ambient air; detecting, by the sensor elements, one or more flammable gas within the ambient air; sending, via a 3.5 mm plug connector of the sensor module, the detected information from the sensor elements to a mobile device; receiving, by an application on the mobile device via a 3.5 mm plug input of the mobile device, the detected information from the sensor module; and displaying, via a user interface of the mobile device, at least a portion of the received information from the sensor module.

### BRIEF DESCRIPTION OF THE DRAWINGS

For a more complete understanding of the present disclosure, reference is now made to the following brief description, taken in connection with the accompanying drawings and detailed description, wherein like reference numerals represent like parts.

FIG. 1 illustrates a perspective view of a sensor module according to an embodiment of the disclosure.

FIG. 2 illustrates a perspective view of a sensor module where the housing is transparent, according to an embodiment of the disclosure.

FIG. 3 illustrates a communication system according to an embodiment of the disclosure.

### DETAILED DESCRIPTION

It should be understood at the outset that although illustrative implementations of one or more embodiments are illustrated below, the disclosed systems and methods may be implemented using any number of techniques, whether currently known or not yet in existence. The disclosure should in no way be limited to the illustrative implementations, drawings, and techniques illustrated below, but may be modified within the scope of the appended claims along with their full scope of equivalents.

The following brief definition of terms shall apply throughout the application:

The term “comprising” means including but not limited to, and should be interpreted in the manner it is typically used in the patent context;

The phrases “in one embodiment,” “according to one embodiment,” and the like generally mean that the particular feature, structure, or characteristic following the phrase may be included in at least one embodiment of the present invention, and may be included in more than one embodiment of the present invention (importantly, such phrases do not necessarily refer to the same embodiment);

If the specification describes something as “exemplary” or an “example,” it should be understood that refers to a non-exclusive example;

The terms “about” or “approximately” or the like, when used with a number, may mean that specific number, or alternatively, a range in proximity to the specific number (for example, +/−10%), as understood by persons of skill in the art field; and

If the specification states a component or feature “may,” “can,” “could,” “should,” “would,” “preferably,” “possibly,” “typically,” “optionally,” “for example,” “often,” or “might” (or other such language) be included or have a characteristic, that particular component or feature is not required to be included or to have the characteristic. Such component or feature may be optionally included in some embodiments, or it may be excluded.

Embodiments of the disclosure include systems and methods for detecting flammable gas, and communicating detected information to a user. To improve the reliability of gas detectors to prevent exposure for a user, it may be important to locate a portable gas detector near the user's body. Such portable gas detectors are used in the industrial field, but portable gas detectors are not commonly used by everyday consumers.

Embodiments disclosed herein include an integrated sensor-detector module (e.g. integral type, which for example might not be readily removable but essentially be permanently attached), unlike conventional portable products. The sensor element(s) may be located within a sensor layer rather than in the form of a traditional sensor (which for example might have separate pluggable sensors), for example with the sensor element(s) located on a substrate with electrical connection leads (and perhaps with other elements, such as a processor, power source, etc. located on a separate, main layer) in some embodiments. So in some embodiments, the sensor layer might only comprise the sensor element(s) and minimum support and/or electric connection components (e.g. a substrate for mounting of the sensor element(s) thereon and electrical contacts and/or leads for electrical interaction with the second/main layer). Typically, the sensor layer would not include a processor or power source or memory storage.

The integrated sensor-detector module may comprise two (internal) layers (e.g. within the housing/cover) in some embodiments, for example with a sensor layer (as described above) and a separate main layer/circuit board (and the two layers may interact only via electric leads in some embodiments). The main layer may comprise in some embodiments a substrate (e.g. separate from the substrate of the sensor layer), a processor (e.g. mounted thereon), electrical contacts and/or leads (e.g. for electrical communication/connection with the sensor layer and/or electrical connection/communication to the connector for the mobile device), and in some embodiments the processor may be configured to communicate with the mobile device via the connector and/or the sensor elements via the electric leads (e.g. acting as a translator between the mobile device and the sensor element(s)). The main layer might be configured in some embodiment to control/operate the senor layer/element(s). In some embodiments, the sensor layer may be permanently attached to the main layer and/or permanently mounted within the filtering housing/cover (as described below). The sensor layer may comprise multiple different combustible gas sensor elements (e.g. for detecting a plurality of different combustible gases), and the user might in some embodiments be able to select which sensor(s) would be active (e.g. by interfacing via the mobile device (see below) through the connector to the second/main layer (which for example might control the sensor elements through the electric leads connecting the main layer to the sensor layer).

In addition, to prevent explosion, at least a portion of the cover, but optionally the entire cover of the sensor module, may comprise a sintered filter, providing high stability. Sintering is the process of compacting and forming a solid mass of material by heat and/or pressure without melting it to the point of liquefaction. Sintering may happen naturally in mineral deposits or as a manufacturing process used with metals, ceramics, plastics, and other materials. Sintering may be effective when the process reduces the porosity and enhances properties such as strength, electrical conductivity, translucency, and thermal conductivity. In other cases, it may be useful to increase a material's strength but keep its gas absorbency constant, as in filters or catalysts. So for example, embodiments may have a sintered filter cover/housing configured to allow the combustible gas being detected to enter into the cover/housing, while also preventing and/or containing any explosion within the cover/housing (for example, ensuring that any combustion/explosion associated with detection of the gas(es) does not inadvertently trigger a wider explosion). The sintered filter cover/housing would typically be configured to provide/allow sufficient combustible gas flow into the cover/housing (e.g. via porosity) while having sufficient mechanical strength and/or porosity capability to contain/limit an internal explosion (e.g. with such mechanical strength sufficient based on likely strength of explosion based on internal volume (e.g. amount of combustible gas within the housing/cover), type of gas, type of reaction at sensor element, etc.). The sintered cover/housing may have porosity sufficient to slow/restrict/prevent outward movement of heat and/or particles in the event of an internal explosion within the housing, so as to prevent propagation of the explosion outward beyond the housing/cover. The sensor element(s) may be configured to be sufficiently sensitive to effectively detect combustible gas(es) even at the limited rate of entrance afforded by the porosity of the sintered filter cover/housing.

The sensor module may be configured to interface with a user's mobile device, making the use of the sensor module convenient for the user. The sensor module may comprise a headphone plug connector, or 3.5 mm plug connector, configured to be inserted into the headphone jack of a user's mobile device. The connector may also be called an audio plug, audio jack, earphone jack, audio terminal, or other similar terminology. The mobile deice might run an application that decodes the signal from the main layer and displays information for user review and/or allows user to control the sensor (e.g. with the application translating/coding user commands for transmission through the connector to the main layer).

The sensor layer of the sensor module may be separate from a main layer. The sensor layer may be provided power by main layer (or detector layer). The main layer may output a signal via the headphone plug connector. The mobile device may comprise a mobile application configured to receive the signal from the sensor module and display information via the mobile device.

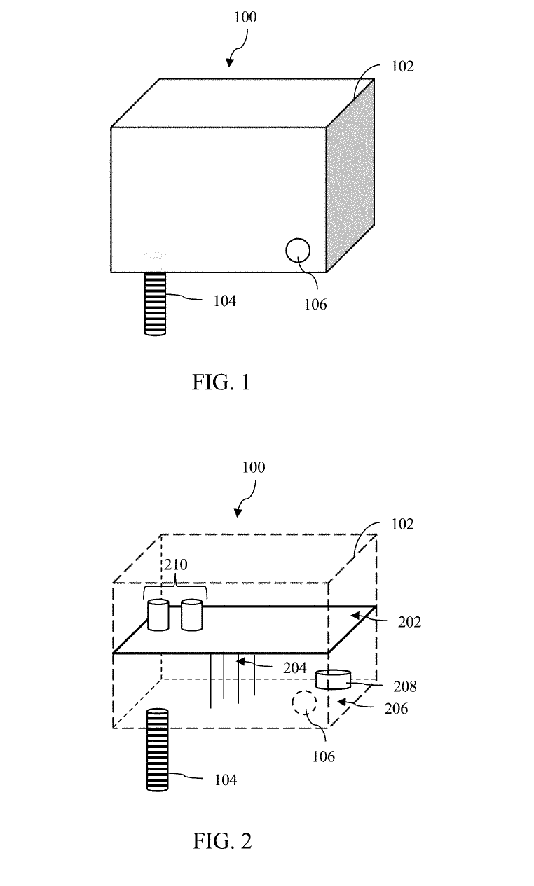

Referring now to FIG. 1, an exemplary sensor module 100 is shown. The sensor module 100 may comprise a housing 102 and a connector 104. At least a portion of the housing 102 may comprise sintered filters, allowing airflow or gas diffusion into the housing 102 while preventing an explosion occurring within the housing 102 from propagating outwards to the environment. The connector 104 may comprise a 3.5 mm plug connector configured to fit into headphone jack of a mobile device or smartphone. In some embodiments, the sensor module 100 may comprise an indicator 106 configured to indicate the status of the sensor module 100. In some embodiments, the indicator 106 may comprise a light-emitting diode (LED).

FIG. 2 shows the sensor module 100 (e.g. similar to FIG. 1) where the housing 102 is shown transparent. The sensor module 100 may comprise a multi-layer construction. One or more sensor elements 210 may be located on a first layer 202 (or “sensor layer”). Additional electrical elements may be located on a second layer 206 (or “main layer”), where one or more leads 204 may connect the first layer 202 and the second layer 206. The connector 104 may be in communication with the second layer 206. In some embodiments, the sensor module 100 may comprise a battery 208 or other power source. In an alternative embodiment, the sensor module 100 may be configured to draw power via the connector 104. In some embodiments, the sensor elements 210 may comprise pellistor elements (e.g., at least one catalytic pellistor element, at least one matched non-catalytic pellistor element, etc.). In some embodiments, the sensor elements 210 may be configured to detect one or more flammable gases, and/or a group of gases.

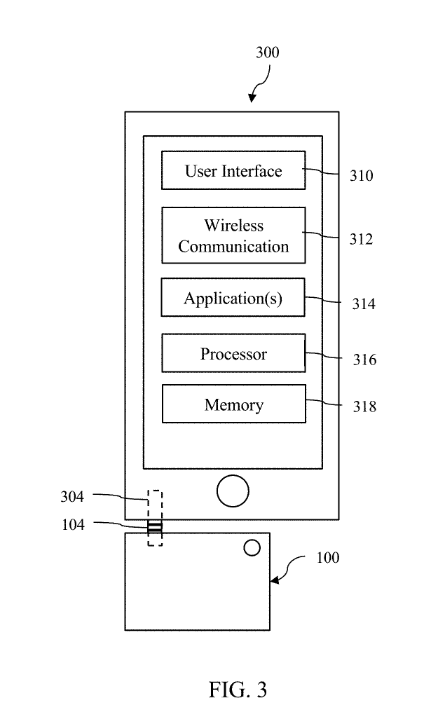

Referring to FIG. 3, the sensor module 100 (e.g. similar to FIG. 1-2) may be used with a mobile device 300, which may comprise a user interface 310, a wireless communication module 312, one or more applications 314, a processor 316, and a memory 318. The mobile device 300 may comprise an input 304 configured to receive the connector 104 of the sensor module 100. In some embodiments, the input 304 may comprise a headphone jack, and/or a 3.5 mm plug connector.

The user interface 310 may comprise a display, an input system, a speaker system, and/or a microphone. The wireless communication module 312 may comprise cellular communication, Wi-Fi communication, Bluetooth communication, radio frequency (RF) communication, near field communication (NFC), or any other wireless communication protocol. The one or more applications 314 may be stored by the memory 318 and executed by the processor 316.

In some embodiments, the one or more applications 314 may comprise an application 314 configured to receive information from the sensor module 100, process the received information, and display the processed information via the user interface 310 of the mobile device 300. For example, the sensor module 100 may provide a voltage or current output indicative of a concentration of one or more gases. The application 314 may be configured to convert the voltage and/or current into a gas concentration using an algorithm, correlation equation, correlation table, or the like. The application 314 may be configured to display the current concentration detected by the sensor module 100. The application 314 may be configured to store received information to the memory 318 of the mobile device 300. The application 314 may be configured to display any alarms, alerts, and/or faults determined by the sensor module 100. The application 314 may be configured to determine when an alarm, alert, and/or fault should be activated based on the received information from the sensor module 100. For example, the application 314 can store or receive an indication of the alarm limits and compare the alarm limits to the measure of the concentration of one or more gases. When the concentration of the one or more gases is outside an expected range or exceeds a threshold, an alarm can be triggered.

Some method embodiments (e.g. for configuring/forming a sensor device and/or system) might comprise one or more of the following steps: providing a sintered filter housing/cover (which can be configured as described above); providing a sensor layer and a main layer (e.g. which can be as described above); electrically interconnecting the sensor layer and the main layer (e.g. using electric leads therebetween); placing/locating the layers within the housing/cover (e.g. so that the housing/cover can prevent propagation of an internal explosion outward); selecting sensor element(s) with respect to the housing (e.g. sufficiently sensitive to the combustible gas(es) to detect the gas(es) at the rate of entrance through the porosity of the filter housing); selecting the porosity of the housing (e.g. sufficient for entrance of combustible gas for effective detection by the sensor(s) and/or sufficient to prevent explosion propagation (e.g. by maintaining sufficient strength to contain an explosion of the level that might occur in the housing based on the factors present and possibly a safety factor and/or by restricting outward flow of heat and/or particles that might ignite combustible gas in the external environment in proximity to the exterior of the housing/cover); selecting/configuring the housing/cover with sufficient strength to contain any explosion; providing a connector plug configured for use with a mobile device and electrically connecting the connector to the main layer; connecting the connector to a mobile device (e.g. to allow communication between the main layer and the mobile device); uploading an application/software to the mobile device to allow for effective decoding/communication of signals from the main layer/processor; configuring the housing/cover with a small internal volume (sufficiently small to minimize the strength of any internal explosion to a level containable by the sintered filter housing/cover); using an off the shelf circuit board as the main layer (e.g. to reduce manufacturing costs and/or complexity).

Having described various devices and methods herein, exemplary embodiments or aspects can include, but are not limited to:

In a first embodiment, a sensor module may comprise a housing configured to allow airflow into the interior of the housing; one or more sensor elements configured to detect flammable gas; and a connector configured to connect to an input of a mobile device, and configured to send information to the mobile device.

A second embodiment can include the sensor module of the first embodiment, wherein the connector comprises a 3.5 mm plug connector.

A third embodiment can include the sensor module of the first or second embodiments, wherein the housing at least partially comprises a sintered filter.

A fourth embodiment can include the sensor module of any of the first through third embodiments, further comprising a first layer and a second layer located within the housing, wherein the one or more sensor elements are located on the first layer, and wherein the connector is located proximate to the second layer.

A fifth embodiment can include the sensor module of any of the first through fourth embodiments, wherein the connector comprises an audio jack connector.

A sixth embodiment can include the sensor module of any of the first through fifth embodiments, further comprising one or more electrical elements including a processor and a memory.

In a seventh embodiment, a communication system may comprise a sensor module comprising: a housing comprising a sintered filter; one or more sensor elements configured to detect flammable gas; and a 3.5 mm plug connector; a mobile device comprising; a 3.5 mm plug input configured to receive the 3.5 mm plug connector of the sensor module; a user interface; a processor; a memory; and an application stored by the memory that when executed by the processor is configured to receive, via the 3.5 mm plug input, detected information from the one or more sensor elements of the sensor module; and display, via the user interface, at least a portion of the received information from the sensor module.

An eighth embodiment can include the communication system of the seventh embodiment, wherein the application is further configured to process the received information from the sensor module.

A ninth embodiment can include the communication system of the seventh or eighth embodiment, wherein the application is further configured to display the current concentration detected by the sensor module.

A tenth embodiment can include the communication system of any of the seventh through ninth embodiments, wherein the application is further configured to store to the memory the received information from the sensor module.

An eleventh embodiment can include the communication system of any of the seventh through tenth embodiments, wherein the application is further configured to display any alarms, alerts, and/or faults received from the sensor module.

A twelfth embodiment can include the communication system of any of the seventh through eleventh embodiments, wherein the application is further configured to determine when an alarm, alert, and/or fault should be activated based on the received information from the sensor module.

A thirteenth embodiment can include the communication system of any of the seventh through twelfth embodiments, wherein the application is further configured to display any alarms, alerts, and/or faults determined by the application.

In a fourteenth embodiment, a method for detecting flammable gas may comprise powering one or more sensor elements of a sensor module; contacting the one or more sensor elements with ambient air; detecting, by the sensor elements, one or more flammable gas within the ambient air; sending, via a 3.5 mm plug connector of the sensor module, the detected information from the sensor elements to a mobile device; receiving, by an application on the mobile device via a 3.5 mm plug input of the mobile device, the detected information from the sensor module; and displaying, via a user interface of the mobile device, at least a portion of the received information from the sensor module.

A fifteenth embodiment can include the method of the fourteenth embodiment, further comprising processing, by the mobile device, the received information from the sensor module.

A sixteenth embodiment can include the method of the fourteenth or fifteenth embodiments, further comprising displaying, by the application, the current concentration detected by the sensor module.

A seventeenth embodiment can include the method of any of the fourteenth through sixteenth embodiments, further comprising storing, by a memory of the mobile device, the received information from the sensor module.

An eighteenth embodiment can include the method of any of the fourteenth through seventeenth embodiments, further comprising determining by the sensor module when an alarm, alert, and/or fault is indicated; and displaying, by the mobile device, any alarms, alerts, and/or faults determined by the sensor module.

A nineteenth embodiment can include the method of any of the fourteenth through eighteenth embodiments, further comprising determining, by the application of the mobile device, when an alarm, alert, and/or fault should be activated based on the received information from the sensor module.

A twentieth embodiment can include the method of any of the fourteenth through nineteenth embodiments, further comprising displaying, by the mobile device, any alarms, alerts, and/or faults determined by the application.

While various embodiments in accordance with the principles disclosed herein have been shown and described above, modifications thereof may be made by one skilled in the art without departing from the spirit and the teachings of the disclosure. The embodiments described herein are representative only and are not intended to be limiting. Many variations, combinations, and modifications are possible and are within the scope of the disclosure. Alternative embodiments that result from combining, integrating, and/or omitting features of the embodiment(s) are also within the scope of the disclosure. Accordingly, the scope of protection is not limited by the description set out above, but is defined by the claims which follow that scope including all equivalents of the subject matter of the claims. Each and every claim is incorporated as further disclosure into the specification and the claims are embodiment(s) of the present invention(s). Furthermore, any advantages and features described above may relate to specific embodiments, but shall not limit the application of such issued claims to processes and structures accomplishing any or all of the above advantages or having any or all of the above features.

Additionally, the section headings used herein are provided for consistency with the suggestions under 37 C.F.R. 1.77 or to otherwise provide organizational cues. These headings shall not limit or characterize the invention(s) set out in any claims that may issue from this disclosure. Specifically and by way of example, although the headings might refer to a “Field,” the claims should not be limited by the language chosen under this heading to describe the so-called field. Further, a description of a technology in the “Background” is not to be construed as an admission that certain technology is prior art to any invention(s) in this disclosure. Neither is the “Summary” to be considered as a limiting characterization of the invention(s) set forth in issued claims. Furthermore, any reference in this disclosure to “invention” in the singular should not be used to argue that there is only a single point of novelty in this disclosure. Multiple inventions may be set forth according to the limitations of the multiple claims issuing from this disclosure, and such claims accordingly define the invention(s), and their equivalents, that are protected thereby. In all instances, the scope of the claims shall be considered on their own merits in light of this disclosure, but should not be constrained by the headings set forth herein.

Use of broader terms such as “comprises,” “includes,” and “having” should be understood to provide support for narrower terms such as “consisting of,” “consisting essentially of,” and “comprised substantially of.” Use of the terms “optionally,” “may,” “might,” “possibly,” and the like with respect to any element of an embodiment means that the element is not required, or alternatively, the element is required, both alternatives being within the scope of the embodiment(s). Also, references to examples are merely provided for illustrative purposes, and are not intended to be exclusive.

While several embodiments have been provided in the present disclosure, it should be understood that the disclosed systems and methods may be embodied in many other specific forms without departing from the spirit or scope of the present disclosure. The present examples are to be considered as illustrative and not restrictive, and the intention is not to be limited to the details given herein. For example, the various elements or components may be combined or integrated in another system or certain features may be omitted or not implemented.

Also, techniques, systems, subsystems, and methods described and illustrated in the various embodiments as discrete or separate may be combined or integrated with other systems, modules, techniques, or methods without departing from the scope of the present disclosure. Other items shown or discussed as directly coupled or communicating with each other may be indirectly coupled or communicating through some interface, device, or intermediate component, whether electrically, mechanically, or otherwise. Other examples of changes, substitutions, and alterations are ascertainable by one skilled in the art and could be made without departing from the spirit and scope disclosed herein.

What is claimed is:

1. A sensor module comprising:
a housing configured to allow airflow into the interior of the housing;
one or more sensor elements configured to detect flammable gas; and
a connector configured to connect to an input of a mobile device, and configured to send information to the mobile device.

2. The sensor module of claim 1, wherein the connector comprises a 3.5 mm plug connector.

3. The sensor module of claim 1, wherein the housing at least partially comprises a sintered filter.

4. The sensor module of claim 1, further comprising a first layer and a second layer located within the housing, wherein the one or more sensor elements are located on the first layer, and wherein the connector is located proximate to the second layer.

5. The sensor module of claim 1, wherein the connector comprises an audio jack connector.

6. The sensor module of claim 1, further comprising one or more electrical elements including a processor and a memory.

7. A communication system comprising:
a sensor module comprising:
a housing comprising a sintered filter;
one or more sensor elements configured to detect flammable gas; and
a 3.5 mm plug connector;
a mobile device comprising;
a 3.5 mm plug input configured to receive the 3.5 mm plug connector of the sensor module;
a user interface;
a processor;
a memory; and
an application stored by the memory that when executed by the processor is configured to receive, via the 3.5 mm plug input, detected information from the one or more sensor elements of the sensor module; and display, via the user interface, at least a portion of the received information from the sensor module.

8. The communication system of claim 7, wherein the application is further configured to process the received information from the sensor module.

9. The communication system of claim 7, wherein the application is further configured to display the current concentration detected by the sensor module.

10. The communication system of claim 7, wherein the application is further configured to store to the memory the received information from the sensor module.

11. The communication system of claim 7, wherein the application is further configured to display any alarms, alerts, and/or faults received from the sensor module.

12. The communication system of claim 7, wherein the application is further configured to determine when an alarm, alert, and/or fault should be activated based on the received information from the sensor module.

13. The communication system of claim 7, wherein the application is further configured to display any alarms, alerts, and/or faults determined by the application.

14. A method for detecting flammable gas, the method comprising:
powering one or more sensor elements of a sensor module;
contacting the one or more sensor elements with ambient air;
detecting, by the sensor elements, one or more flammable gas within the ambient air;
sending, via a 3.5 mm plug connector of the sensor module, the detected information from the sensor elements to a mobile device;
receiving, by an application on the mobile device via a 3.5 mm plug input of the mobile device, the detected information from the sensor module; and
displaying, via a user interface of the mobile device, at least a portion of the received information from the sensor module.

15. The method of claim 14, further comprising processing, by the mobile device, the received information from the sensor module.

16. The method of claim 14, further comprising displaying, by the application, the current concentration detected by the sensor module.

17. The method of claim 14, further comprising storing, by a memory of the mobile device, the received information from the sensor module.

18. The method of claim 14, further comprising determining by the sensor module when an alarm, alert, and/or fault is indicated; and displaying, by the mobile device, any alarms, alerts, and/or faults determined by the sensor module.

19. The method of claim 14, further comprising determining, by the application of the mobile device, when an alarm, alert, and/or fault should be activated based on the received information from the sensor module.

20. The method of claim 14, further comprising displaying, by the mobile device, any alarms, alerts, and/or faults determined by the application.
